(19) United States
(12) Patent Application Publication
(10) Pub. No.: US 2003/0229357 A1
(43) Pub. Date: Dec. 11, 2003

(54) Femoral head holder and impaction instrument and method of use

(76) Inventor: Donald Dye, Pflugerville, TX (US)

Correspondence Address: Attention: Kenneth S. Barrow, Centerpulse USA Inc., Suite 1000, 12 Greenway Plaza, Houston, TX, 77046, US

(21) Appl. No.: 10/375,480

(22) Filed: Feb. 27, 2003

Related U.S. Application Data
(60) Provisional application No. 60/387,360, filed on Jun. 10, 2002.

(51) Int. Cl. A61B 17/74

(52) U.S. Cl. 606/99

(57) ABSTRACT

A method and apparatus for performing minimally invasive hip replacement surgery using a femoral head holder and impaction instrument to attach and then impact a femoral head onto a femoral implant embedded into the intramedullary canal of the femur. The instrument includes a driver and holder. The holder captures a femoral head and positions it on the neck of the femoral implant. The driver is then impacted to insert the head onto the implant.

### CROSS-REFERENCE TO RELATED APPLICATIONS

This application claims benefit of priority of U.S. Provisional Application Serial No. 60/387,360 filed Jun. 10, 2002.

### FIELD OF THE INVENTION

The disclosure herein generally relates to a method and apparatus for performing minimally invasive hip replacement surgery using a femoral head holder and impaction instrument to attach and then impact a femoral head onto a femoral implant embedded into the intramedullary canal of the femur.

### BACKGROUND OF THE INVENTION

Traditional hip replacement surgery has been used in the United States since as early as the 1960's. The surgical technique to implant a hip has not drastically changed over the years, and today, this technique is quite successful. In fact, the surgical technique is prolifically used throughout the world and has a known success rate of over 90%. Certainly, the traditional surgical technique is fundamentally sound and predictable.

Unfortunately, traditional techniques to implant a hip have well recognized shortcomings. Most importantly, a rather large incision is made on the side of the hip. The incision can extend from 6 to 12 inches; the actual length of the incision depends on the size of the patient and type of surgery (revision versus total hip arthroplasty, for example). A long, deep incision can divide a number of important stabilizing muscles and tendons and further damage the hip joint and surrounding soft tissue. Inevitably, long incisions lead to larger blood losses, longer rehabilitation times for patients, and unsightly scar lines. A patient can easily spend four or five days in the hospital after a total hip arthroplasty, for example.

Recently, surgeons have been developing new, less invasive surgical techniques to perform total hip arthroplasty and revision hip surgery. Minimally invasive surgery, or MIS, is one such technique with great promise to become a popular and accepted technique for implanting a hip.

MIS has significant advantages over traditional hip replacement surgery. Most importantly, a rather small incision is made on the side on the hip. This incision is approximately 3 to 5 inches long, and the benefits of a shorter incision are enormous.

First and foremost, the patient can recover in a much shorter period of time after a MIS. The recuperation time in the hospital can be a few days and significantly reduce the cost to both the patient and hospital. In fact, some patients are leaving the hospital within 24 to 48 hours after the surgery. Obviously, this shortened time period is extremely important to the patient.

As another advantage, MIS is less invasive and traumatic to the patient. Significantly less soft tissue is disrupted in a minimally invasive surgery compared to a traditional hip surgery. Also, the amount of blood loss is reduced, and patients will require fewer blood transfusions. Further, the length of the scar is significantly smaller, and these scars are more cosmetically appealing. The incisions themselves heal in a much shorter period of time and are much less painful than a long ten or twelve inch incision. As such, the patient can sooner return to work or enjoy recreational activities. In short, the patient can more quickly return to a normal way of life.

Presently, instruments to perform MIS are being developed and refined. These instruments have a vital role in the ability to perform a successful minimally invasive surgery. These instruments, for example, must enable the surgeon to place the hip implant in a very precise location. If the implant is not accurately placed, then complications, such as dislocation or subluxation, can occur. Further and most importantly, the instruments must consistently and reliably perform through a small three inch opening in the patient.

A successful design of instruments for MIS has other challenges as well. Specifically, the instrument must be easy to use and facilitate the implantation procedure. If the MIS instrumentation is too cumbersome or not easy to manipulate, then the surgeon will be less likely to use minimally invasive surgery. The patient, then, will not reap the benefits MIS has to offer.

As yet another consideration, MIS instrumentation must appeal to a wide range of orthopedic surgeons with various skills and experience. If, for example, the instruments are too complex and complicated, then they will not be appealing and accepted in the orthopedic surgical community. Further yet, the training and skill level required to use the instruments and become proficient with them, cannot be overly taxing on the orthopedic surgeons.

While implanting or repairing a prosthetic femoral prosthesis in MIS for instance, a femoral head must be attached to a neck that extends outwardly from the prosthesis. The head must be positioned onto the neck and then impacted to drive the head onto the neck. Traditional head impaction instruments, though, are not shaped and sized to perform the multiple steps necessary to connect a head to the femoral prosthesis. In order to perform these steps, several different instruments and procedures are needed. For example, the surgeon must grasp the femoral head or head trial with his/her fingers in order to insert the femoral head or trial into the wound. During this time, great care must be taken not to accidentally drop the femoral head or trial into the wound. Once the femoral head or trial is positioned on the neck, a separate instrument is required to impact the head onto the femoral prosthesis. In this example, two separate and distinct procedures are required to connect and impact a femoral head to a prosthetic femur.

A great advantage would be realized if a single instrument could perform the multiple steps necessary to attach and impact a femoral head onto a prosthetic femur embedded in the intramedullary canal of a femur. A significant cost savings would occur since the overall number of instruments required to perform the surgery is reduced. Further, a significant time savings would occur since less surgical steps would be required to attach and impact the femoral head to the prosthesis. Other benefits as well would be realized from a surgical instrument that performed multiple functions for MIS.

In short, instruments, and in particular instruments for attaching, positioning, removing, and impacting a femoral head to the prosthesis, play a vital role in MIS surgery for hip implantation. It therefore would be advantageous to provide a new method and accompanying instrument for attaching and impacting a femoral head to a femoral prosthesis in minimally invasive surgery.

### SUMMARY OF THE INVENTION

The present invention is directed to a method and apparatus for performing minimally invasive hip replacement surgery using a femoral head holder and impaction instrument to position, attach, and then impact a femoral head onto a femoral implant embedded into the intramedullary canal of the femur. The instrument can also remove a femoral head once it is placed on the femoral prosthesis.

The method of the present invention generally comprises the steps of templating the side of the femur to be reconstructed; incising the surgical site with a minimally invasive incision from about 2½ inches to about 5 inches in length; providing retractors to retract soft tissue; dislocating the hip from the acetabulum; transecting the femoral neck of the femur; reaming and broaching the intramedullary canal; placing a planar over a broach to plane the calcar; implanting a femoral prosthesis; providing a femoral head holder and impaction instrument; attaching a trial head to the femoral prosthesis using the instrument; removing the trial head from the prosthesis and from the instrument; attaching the final head to the instrument; inserting the final head and instrument into the surgical site; positioning the final head on the neck of the femoral prosthesis; impacting an end of the instrument to drive the final head onto the neck of the femoral prosthesis; detaching the instrument from the final head; removing all instruments from the surgical site; and closing the surgical site.

The instrument generally comprises two main components, a driver and a holder. The driver has a handle with an impaction end at one end and an elongated shaft extending from another end. The shaft has a distal threaded tip adapted to threadably engage the holder. The holder has a body with a conical shape that forms an internal socket. This socket is sized and shaped to receive a femoral head. A plurality of radially flexible fingers circumferentially extend around the body to form the socket. A relief cut is formed in the body where no fingers are present. A proximal end of the body includes a threaded bore adapted to threadably receive the threaded tip of the shaft.

In use, a femoral head is pushed or forced into the socket. As the head and holder move together, the fingers radially expand and allow the head to snap into the socket. In this position, the head is captured in the holder. Further, the head may move while capture in the socket. In order to disengage the head from the socket, the head and the holder are pulled or moved away from each other. The fingers radially flex outwardly and snappingly disengage from the head.

One important advantage of the present invention is that the method and instrument are used in a minimally invasive orthopedic hip surgery. A single, small three to five inch incision is made at the surgical site on the side on the hip. The method of the present invention, thus, enjoys the benefits of a shorter incision compared to traditional hip surgery that uses a much longer incision. As one benefit, the patient can recover in a much shorter period of time after a MIS. The recuperation time in the hospital can be a few days and significantly reduce the cost to both the patient and hospital. This shortened time period is extremely important to the patient. Further, MIS is less invasive and traumatic to the patient. Significantly less soft tissue is disrupted in a minimally invasive surgery compared to a traditional hip surgery. Also, the amount of blood loss is reduced, and patients will require fewer blood transfusions. Further, the length of the scar is significantly smaller, and these scars are more cosmetically appealing. The incisions themselves heal in a much shorter period of time and are much less painful than a long ten or twelve inch incision. As such, the patient can sooner return to work or enjoy recreational activities. In short, the patient can more quickly return to a normal way of life.

Another important advantage of the present invention is that the femoral head holder and impaction instrument performs several important functions. First, the instrument engages, holds, carries, and aligns a femoral head to the neck of a femoral prosthesis embedded in the intramedullary canal of the patient. Second, the instrument impacts or drives the head onto the neck. Third, the instrument can remove the femoral head from the prosthesis if such removal is necessary.

Importantly, the same instrument performs both of the noted functions. A great advantage is, thus, realized since a single instrument can perform the multiple steps necessary to attach and impact a femoral head onto a prosthetic femur embedded in the intramedullary canal of a femur. A significant cost savings occurs since the overall number of instruments required to perform the surgery is reduced. Further, a significant time savings occurs since less surgical steps are required to attach and impact the femoral head to the prosthesis.

Further yet, the surgeon is not required to grip the femoral head or trial head with his/her fingers in order to insert the head into the wound. In this instance, the surgeon could accidentally drop the head into the wound.

Another important advantage of the present invention is that the holder provides an audible sound when the socket snappingly engages the femoral head or trial head. This sound is particularly important in MIS since the surgeon may not be able to visually verify if the femoral head is fully seated on the neck of the femoral prosthesis.

Another advantage of the present invention is that the holder has a tapering or conical body. This shape is important because extraneous material has been removed from the body. As such, the holder is adapted to be used in MIS and inserted through the small incision.

Another advantage of the present invention is that the holder firmly captures the femoral head in the socket. As such, the likelihood that the femoral head will fall off or prematurely disengage from the holder is greatly reduced. This feature is particularly important in MIS since the holder and femoral head are positioned through a tight, small incision.

Another important advantage of the present invention is that the holder must unsnap from the femoral head before disengaging from the head while it is connected to the prosthesis. In order to unsnap the holder from the head, the holder is pulled away from the head in a direction generally parallel to the central axis of the neck. If the head is not fully seated on the neck, then the head will remain captured in the holder as it is pulled away. On the contrary, if the head is fully seated on the neck, then the head will remain on the neck as the holder pulls away and produces a “snap” sound with an accompanying tactile feedback sensation that can be sensed while holding the instrument. The snap sound provides a security check for the surgeon that the femoral head is fully seated on the neck. The snap is particularly advantageous when the holder engages the femoral head or trial head. For example, while the trial head is positioned on the femoral prosthesis, the wound channel obstructs the view of the trial head. The surgeon can verify that the holder connects to the trial head when a snapping sound and sensation occurs.

As another advantage, the instrument can consistently and reliably perform through a small three to five inch opening in the patient. Importantly, the length of the shaft of the driver is specifically adapted to pass through a small incision and reach an embedded neck of a femoral prosthesis.

Further yet, the instrument is easy to use and facilitates the implantation procedure. The driver readily attaches and detaches from the holder. As such, use of the instrument can appeal to a wide range of orthopedic surgeons with various skills and experience. Further yet, the training and skill level required to use the instrument and become proficient with it is not overly taxing on the orthopedic surgeon.

### BRIEF DESCRIPTION OF THE DRAWINGS

FIG. 1 is a sketch of a patient showing a femur and femoral head positioned in the acetabulum with an MIS incision marked along the hip.

FIG. 2 is a side view of the femoral head holder and impaction instrument.

FIG. 3 is a side view of the driver of the instrument.

FIG. 4 is a side view of the holder of the instrument.

FIG. 5 is a top view of the holder of the instrument.

FIG. 6 is a cross sectional view taken through the lines A-A of FIG. 5.

FIG. 7 is an exploded perspective view of the holder of the instrument.

FIG. 8 is a perspective view of a holder holding a femoral ball adjacent a neck of a femoral prosthesis embedded in the intramedullary canal of a femur of a patient.

FIG. 9 is a perspective view of a holder holding a femoral ball onto a neck of a femoral prosthesis embedded in the intramedullary canal of a femur of a patient.

### DETAILED DESCRIPTION

The instruments, method, and steps of the present invention are now described in more detail. The method describes the steps to perform a minimally invasive surgery to implant a prosthetic femoral hip stem in the intramedullary canal of a natural femur. Some of these steps described in the method are known to those skilled in the art and will not be discussed in great detail. Further, one skilled in the art will appreciate that certain steps may be altered or omitted while other steps may be added without departing from the scope of the invention. The novel steps of the present invention, for example, can be applied to total hip arthroplasty, to revision surgeries for total and partial hip replacement, and to other orthopedic hip surgeries using minimally invasive surgical techniques.

To facilitate a discussion of the present invention, the method of implanting a prosthetic femoral hip stem is divided into a plurality of steps or sections. Each of these sections is discussed seriatim.

More specifically, the method of the present invention teaches how to use a femoral head holder and impaction instrument to attach and then impact a femoral head onto a femoral implant embedded into the intramedullary canal of the femur. For illustrative purposes, the discussion focuses on implanting a Natural™ Hip System of Centerpulse Orthopedics Inc. of Austin, Tex. This system illustrates one possible hip system that can be used. One skilled in the art will appreciate that other, different hip systems can also be used with the method and apparatus of the present invention without departing from the scope of the invention.

Templating the Femur

Typically, the side of the femur to be reconstructed is templated. Use of a template enables the surgeon to make an estimation of the size of instruments, trials, and prosthetic components to be used during the surgical procedure. Templating and other preoperative techniques will also help to identify bone abnormalities and other potential problems before the surgery. Further, these procedures aid the surgeon in restoring the center of rotation of the hip and placing the femoral prosthesis with the correct length and offset. The exact procedures for templating the femur are known in the art and will not be discussed in detail

Incising the Surgical Site

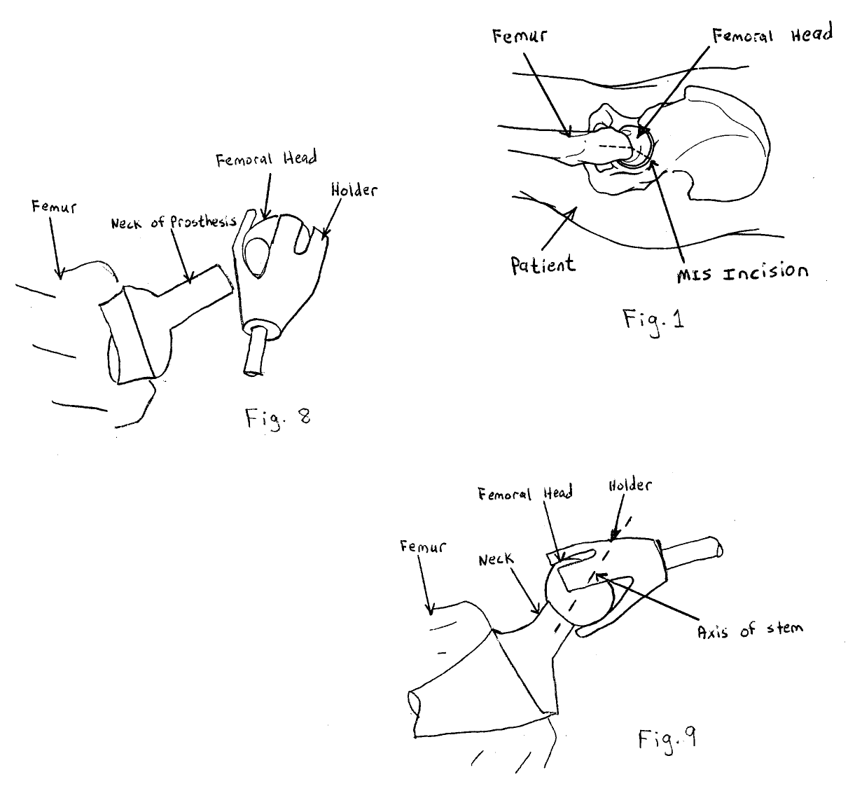

As shown in FIG. 1, a relatively small, single minimally invasive incision is made at the surgical site. A minimally invasive incision for this procedure has a length from about 2½ inches to about 4 or 5 inches. The incision is slightly curved or straight, commences near the vastus tubercle, and continues toward the greater trochanter and posterior inferior spine. The incision should be carried down through subcutaneous tissue and fascia lata. Any muscle tissue should be gently split in line with its fibers. At this time, a leg length measurement can be taken using techniques known in the art.

Providing Retractors

The retractors have an elongated, flat, thin body with two primary sections, a handle section and a retracting section. The handle section is elongated and adapted to be gripped with a hand. A smooth curved section transitions the handle section to the retracting section. The retracting section typically has a paddle and may further include a prong.

Dislocating the Hip from the Acetabulum and Transecting the Femoral Neck

Next, dislocation of the hip occurs. A bone hook or skid may be used to avoid excess torsion on the femoral shaft. Also, retractors may be placed at various locations, for example under the femoral head or lesser trochanter, in order to achieve better visualization for proper transection of the femoral neck at the templated level. Care should be taken to protect the sciatic nerve.

Preparing the Intramedullary Canal

After completing preparation of the acetabulum, the femur may be rotated for a better position, and retractors may be used to lift the femur and retract posterior soft tissue.

Proportional sized tapered reamers with blunt tips are used to prepare the intramedullary canal. The reamers have a calcar stop to assure proper depth of penetration. Generally, reaming occurs laterally against the greater trochanter to ensure that the reamer enters the canal in a neutral position. Each reamer can be followed with a correspondingly sized broach. The broach has cutting teeth to help prepare the canal. This sequence of reaming and broaching continues until the next to the last templated size broach is inserted. This next to last broach is used for the calcar planing process.

A calcar planar is placed over the proximal broach, and the calcar is planed flat to allow proper seating of the collar on the stem. Once calcar planing is finished, the final reamer and broach sequence is performed. The final broach should fill the proximal region and be stable. The final broach can be used to trial the appropriate head/neck adaptor.

Implanting the Femoral Prosthesis

Proper size of the femoral prosthesis is based on the size of the final broach. Once an implant is selected, it is attached to a femoral implant holder. The holder assists in controlling rotation and enables the implant to be inserted into the intramedullary canal with proper anteversion. A mallet or slaphammer can be used to impact the implant and fully seat it to a final position with the collar contacting the calcar.

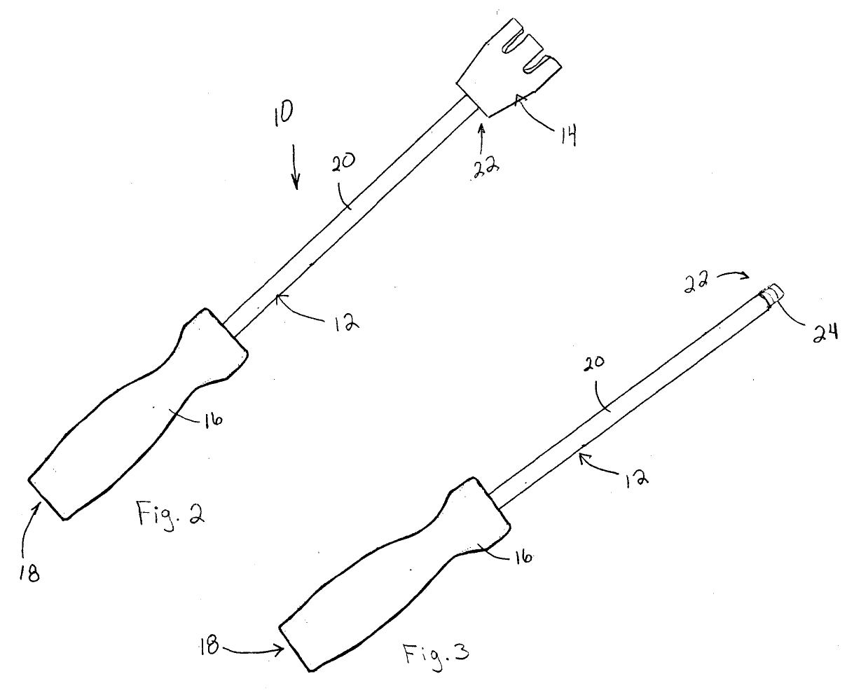

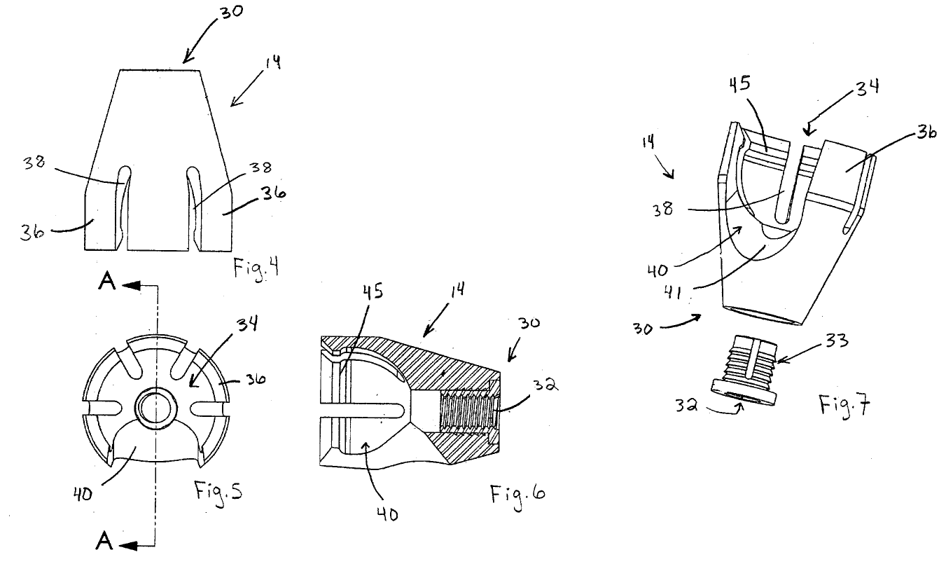

Providing a Femoral Head Holder and Impaction Instrument (See FIGS. 2-7)

A femoral head holder and impaction instrument is provided. This instrument includes two major components, a driver and a holder. The driver has a handle and a shaft that extends outwardly from the handle. A distal end of the shaft includes external threads. The holder has a body with a conical shape that forms an internal socket adapted to receive and hold a femoral head. A proximal end of the body includes a threaded bore adapted to receive the external threads on the driver. The body further includes a plurality of flexible fingers that extend outwardly to form the socket. A relief cut is formed in the body where several finger have been removed. The instrument is discussed in more detail with reference to the drawings.

Attaching a Trial Head to the Femoral Prosthesis (See FIGS. 8-9)

A femoral trial head is provided and attached the holder of the instrument. Specifically, the head is snapped into the socket of the holder with the open face of the trial head facing backwards and out through the relief cut. A “snapping” sound occurs when the holder and head connect together. The holder is threadably connected to the driver, if this step has not yet been completed.

Next, the trial head and holder are positioned through the surgical site until the opening of the trial head aligns with the neck of the femoral prosthesis. The handle of the instrument is then pulled downwardly or toward the distal portion of the femoral prosthesis so the trial head seats or engages on the trunion taper of the neck. Continue pulling until the holder snaps off of the trial head.

Removing a Trial Head from the Femoral Prosthesis (See FIGS. 8-9)

In order to remove a trial head from the femoral prosthesis, position the socket of the holder approximately perpendicular to or superior to the neck. Push the holder onto the trial head so the socket snaps over the trial head. A “snapping” sound occurs when the holder and head connect together. Next, rotate the instrument toward the distal end of the femoral prosthesis so the relief cut is adjacent the neck. In this position, the trial head is captured inside the holder. Push the instrument upwardly toward the proximal end of the femoral prosthesis in a direction generally parallel with the stem. The trial head should pull off from the stem and be captured in the socket of the holder. Remove the holder and trial head from the surgical site.

Attaching a Final Head to the Femoral Prosthesis (See FIGS. 8-9)

A femoral final head is provided and attached the holder of the instrument. Specifically, the head is snapped into the socket of the holder with the open face of the head facing backwards and out through the relief cut. A “snapping” sound occurs when the holder and head connect together. The holder is threadably connected to the driver, if this step has not yet been completed.

Next, the final head and holder are positioned through the surgical site until the opening of the head aligns with the neck of the femoral prosthesis. The handle of the instrument is then pulled downwardly or toward the distal portion of the femoral prosthesis so the head seats or engages on the trunion taper of the neck.

While the head is still engaged in the socket of the holder, rotate the instrument upwardly toward the final head so the instrument is generally parallel with the axis of the stem. Provide a mallet or similar device and impact the proximal end of the instrument adjacent the handle. Continue to impact the instrument until the head is fully seated on the neck of the femoral prosthesis.

Once the head is fully seated and while the instrument is still generally parallel with the axis of the stem, pull the handle of the instrument away from the head until the socket snaps out of engagement with the head. The snap serves as an audible verification that the head is fully seated on the neck. Remove the holder from the surgical site.

Closing Surgical Site

Once the femoral head is firmly connected to the prosthetic femur, all instruments and devices are removed from the site. The prosthetic femur and femoral head should now be properly positioned. A final inspection of the joint should be made to ensure no residual material or osteophytes are present.

Closure of the site may occur with well known techniques. Further, this disclosure will not discuss post-operative protocol or rehabilitation as such procedures are known in the art and tailored to meet the specific needs of the patient.

Detailed Description of Driving Instrument

One important advantage of the present invention is that the femoral head holder and impaction instrument is specifically designed and adapted to be used in minimally invasive surgical techniques for holding, aligning, and impacting a femoral head onto the neck of a femoral prosthesis embedded into the intramedullary canal of a patient.

FIG. 2 shows an assembled femoral head holder and impaction instrument 10. Looking to FIGS. 2-9, the instrument generally comprises two main components, a driver 12 and a holder 14. The driver 12 has a handle 16 adapted to be gripped with a hand. A proximal end 18 of the handle is formed as an impaction end and is adapted to receive blows or impacts from a surgical hammer, mallet, or the like. An elongated, cylindrical shaft 20 extends outwardly from the handle. This shaft has a distal end 22 with external threads 24.

Holder 14 has a body with a generally conical or frusto-conical shape. A proximal end 30 of the body has an internally threaded bore 32 that is adapted to threadably receive the threads 24 on shaft 20. The bore may be formed as a plug 33 (FIG. 7) that is permanently pressed into the body of the holder. The holder and driver are, thus, removeably connectable to each other. The body forms an internal socket or cavity 34 that is shaped to receive a trial head or final head (FIGS. 8 and 9). A plurality of fingers 36 circumferentially extend around the body to form the socket 34. Gaps 38 are formed between adjacent fingers so the fingers flexibly expand in a radial direction. The body also includes a relief cut 40 having a U-shape or generally rectangular shape with a smooth, tapered transition 41 (FIG. 7) that leads into the socket 34. This relief cut is formed where fingers of the body have been removed.

In use, a femoral head is positioned above or adjacent the socket and then pushed or forced into the socket. As the head and holder move together, the fingers expand and allow the head to snap into the socket. A “snapping” sound occurs when the holder and head connect together. In this position, the head is captured in the holder. Further, the head may move while captured in the socket.

In order to disengage the head from the socket, the head and the holder are pulled or moved away from each other. The fingers radially flex outwardly and snappingly disengage from the head. An unsnapping sensation can be felt while holding the instrument.

The size of the socket may vary, depending on the size of femoral head to be captured. Further, a plurality of holders can be provided with differently sized sockets. Each holder would be adapted to engage with a particularly sized femoral head. For example, the sockets could be provided to engage with 28 mm, 32 mm, and 36 mm femoral heads.

Further, the internal walls of the socket can be adapted to grip the femoral head. These walls, for example, can be provided with inner ribs 45 (FIGS. 6 and 7) or the like to engage and capture a femoral head. Further, one skilled in the art will appreciate that the push-in or pull-out force required to engage or disengage a femoral head from the socket can easily be modified with modifications or variations to different elements of the holder. For instance, changing the radial flexibility of the fingers will alter this force. Further, changing the shape of the socket will alter this force. Further yet, changing the choice of materials for the holder will also alter this force. Other modifications as well can be made to alter this force. As such, one advantage of the invention is that the force to engage or disengage the femoral head and holder can be designed with any one of various modifications to the holder. The instrument, thus, can be designed to meet the specific needs of a prosthetic line of femoral components.

Further yet, although the driver and holder are shown as two separate components removeably connectable to each other, these components can be combined or integrally formed together as one component.

It should be emphasized that although the method of the present invention was described with a specific number and sequence of steps, these steps can be altered or omitted while other steps may be added without departing from the scope of the invention. As such, the specific steps discussed in the preferred embodiment of the present invention illustrate just one example of how to utilize the novel method and steps of the present invention. Further, although illustrative embodiments and methods have been shown and described, a wide range of modifications, changes, and substitutions is contemplated in the foregoing disclosure and in some instances, some features of the embodiments or steps of the method may be employed without a corresponding use of other features or steps. Accordingly, it is appropriate that the appended claims be construed broadly and in a manner consistent with the scope of the embodiments disclosed herein.

What is claimed is:

1. A method for using minimally invasive surgery to attach a femoral head to a prosthetic femur embedded in the intramedullary canal of a patient, comprising the steps of:
incising a hip with a minimally invasive incision to access a neck of the prosthetic femur embedded in the intramedullary canal of the patient;
providing a femoral head holder and impaction instrument having a proximal impaction end and a distal holder;
connecting the femoral head to the holder;
inserting the holder and femoral head through the minimally invasive incision;
using the instrument to position the femoral head onto the neck of the prosthetic femur;
impacting the impaction end of the instrument to drive the femoral head onto the neck;
disengaging the instrument from the femoral head;
removing the instrument from the incision; and
closing the incision.

2. The method of claim 1 wherein the step of incising a hip creates the minimally invasive incision with a length of about 2½ inches to about 4 to 5 inches.

3. The method of claim 2 further comprising the step of providing the step of snapping the holder onto the femoral head to perform the step of connecting the femoral head to the holder.

4. The method of claim 3 further comprising the step of unsnapping the holder from the femoral head to perform the step of disengaging the instrument from the femoral head.

5. The method of claim 2 further comprising the step of rotating the instrument to be generally parallel with an axis of the stem prior to performing the step of impacting the impaction end of the instrument.

6. The method of claim 1 further comprising the step of providing an audible snap sound during the step of connecting the instrument to the femoral head.

7. The method of claim 1 further comprising the steps of:
pulling the instrument in a direction generally parallel with an axis of the stem to perform the step of using the instrument to position the femoral head onto he neck of the prosthetic femur; and
rotating the instrument to be generally along the axis of the stem prior to performing the step of impacting the impaction end.

8. The method of claim 1 wherein the step of connecting the femoral head to the holder further comprises the step of capturing the femoral head into the holder.

9. A method for implanting a prosthetic femoral hip, comprising the steps of:
incising a hip of a patient with an incision having a length of about 2½ inches to 5 inches;
exposing a natural femur of the patient;
transecting a femoral head of the natural femur;
reaming and broaching an intramedullary canal of the femur;
inserting the prosthetic femoral hip into the intramedullary canal;
providing an instrument having a handle with a proximal impaction end and having a holder at a distal end, the holder defining a socket;
connecting a femoral head to the socket of the holder;
inserting the holder and femoral head through the incision;
using the instrument to position the femoral head onto a neck of the prosthetic femoral hip;
impacting the impaction end of the instrument to drive the femoral head onto the neck;
disengaging the instrument from the femoral head;
removing the instrument from the incision; and
closing the incision.

10. The method of claim 9 further comprising the step of providing the holder with a substantially conical shape.

11. The method of claim 9 further comprising the step of providing the holder with a body having a plurality of flexible fingers defining at least a portion of the socket.

12. The method of claim 11 further comprising the steps of:
providing the body with a relief cut; and
positioning the neck into the relief cut to perform the step of connecting a femoral head to the socket of the holder.

13. The method of claim 12 further comprising the step of providing the relief cut with a generally rectangular shape.

14. The method of claim 11 further comprising the step of radially flexing the fingers outwardly to perform the step of connecting a femoral head to the socket of the holder.

15. The method of claim 12 further comprising the step of radially flexing the fingers outwardly to perform the step of disengaging the instrument from the femoral head.

16. The method of claim 9 further comprising the step of providing the instrument with a removeable connection between the handle and the holder.

17. A method for using minimally invasive surgery to attach a femoral head to a prosthetic femur embedded in a femur of a patient, comprising the steps of:
incising a hip with a minimally invasive incision to access a neck of the prosthetic femur embedded in the femur of the patient;
providing a instrument having a proximal impaction end and a distal holder;
connecting the femoral head to the holder;
inserting the holder and femoral head through the minimally invasive incision;
aligning the femoral head onto the neck of the prosthetic femur with the instrument;
impacting the impaction end of the instrument to drive the femoral head onto the neck;
removing the instrument from the incision; and
closing the incision.

18. The method of claim 17 further comprising the step of generating an audible snapping sound while connecting the holder to the femoral head.

19. The method of claim 17 further comprising the step of providing the holder with a conical shape defining a recess adapted to capture the femoral head.

20. The method of claim 19 further comprising the step of providing the holder with at least one flexible finger for capturing the femoral head in the recess.
